(19) United States
(12) Patent Application Publication
(10) Pub. No.: US 2016/0003038 A1
(43) Pub. Date: Jan. 7, 2016

(54) Determining Gas Content of a Core Sample

(71) Applicant: Natural Environment Research Council, Swindon, Wiltshire (GB)

(72) Inventors: David Smith, Dalkeith (GB); Michael Wilson, Edinburgh (GB); Lee Baines, Edinburgh (GB); Rob Ling, Perth (GB)

(73) Assignee: NATURAL ENVIRONMENT RESEARCH COUNCIL, Swindon, Wiltshire (GB)

(21) Appl. No.: 14/762,072

(22) PCT Filed: Jan. 14, 2014
(86) PCT No.: PCT/GB2014/050096

§ 371 Date: Jul. 20, 2015

(30) Foreign Application Priority Data
Jan. 21, 2013 (GB) 1301033.5

(51) Int. Cl. E21B 49/00; E21B 49/02; G01N 33/00; E21B 25/18

(52) U.S. Cl. CPC E21B 49/001; E21B 25/18; E21B 49/025; G01N 33/0036

(57) ABSTRACT

An inner barrel for a core barrel or a core barrel assembly, the inner barrel having one or more side walls bounding at least partially an elongate internal volume for receiving, in use, a collected core sample, wherein the or each side wall is adapted to provide at least one fluid flow path from the elongate internal volume to outside the inner barrel.

The present invention relates to methods of determining the gas content of core samples and apparatus for use in such methods.

Gas hydrates, in particular methane hydrates, are found in reservoirs in subterranean formations, typically in deepwater locations, e.g. under the seabed. At the very low temperatures and very high pressures within these reservoirs, gas hydrates exist in a stable crystalline form. When the temperature increases and/or the pressure decreases, the gas hydrate changes state to a gas, which is accompanied by a massive volume expansion.

This volume expansion can be a significant safety hazard in hydrocarbon production, particularly offshore deepwater hydrocarbon production. Generally, in such operations it may be desired to avoid drilling through any reservoirs containing gas hydrates. However, reservoirs containing gas hydrates are typically located less far below the seabed than traditional oil and gas reservoirs. Accordingly, in order that any reservoirs containing gas hydrates can be avoided, reliable surveying and measuring techniques are required.

Vast amounts of gas, typically methane, are stored as hydrates, particularly in marine sediments and cold regions such as the Arctic. Furthermore, the phase-change behaviour of gas hydrates is becoming better understood. Hence, gas hydrates are now attracting interest as an energy resource. Accordingly, when prospecting or surveying for possible gas hydrate resources, reliable and cost-effective survey techniques are required.

Thus, it may be desirable to economically develop hydrate resources. Such resources are often located in deepwater and/or Arctic areas. However, it can be challenging to find and evaluate shallow gas hydrate deposits, e.g. methane hydrate deposits.

For instance, indirect geophysical methods such as electromagnetic (EM) methods or seismic methods are unreliable, due to the nature of gas hydrates.

Estimating hydrate content based on water freshening may be unreliable, due to uncertainty over baseline porewater salinity.

Measuring the actual hydrate content of cores recovered during drilling offshore has been especially challenging, as known techniques can be unreliable and/or expensive.

It is known to recover core samples and to bring the samples to the surface in pressurised core barrels. A pressurised core barrel is intended to store a core sample at an in-situ pressure and temperature, in order to inhibit decomposition of hydrate crystals due to pressure decrease and/or temperature increase when the core sample is lifted to the surface. The sample can then be analysed at the surface.

However, pressurised core barrels are expensive and can be unreliable. For instance, there is a frequent failure to recover the core sample at a lower than in-situ pressure and/or at a higher than in-situ temperature, which may cause a systematic bias in the reported hydrate content from successful core samples. Accordingly, direct measurement of core data from such core samples may be unreliable. In addition, dissociation of the gas hydrate may even result in failure of the pressurised core barrel as it is being brought up to the surface.

Furthermore, significant health and safety issues arise when handling pressurised containers on the surface. Moreover, space may be limited on an offshore drilling platform or vessel for storing and/or handling a pressurised core barrel, thereby increasing the potential risks.

An alternative method is disclosed in WO 2011/082870. In this method, a methane content of a bottom sample comprising methane hydrate crystals is determined by: taking a core sample from a bottom sediment in a deepwater area; storing the core sample in a storage chamber; lifting the storage chamber to a predetermined waterdepth at which any methane hydrate crystals in the core dissociate into water and methane; and measuring an amount of methane released by the lifted core sample.

A first aspect of the invention provides an inner barrel for a core barrel or a core barrel assembly, the inner barrel having one or more side walls bounding at least partially an elongate internal volume for receiving, in use, a collected core sample, wherein the or each side wall is adapted to provide at least one fluid flow path from the elongate internal volume to outside the inner barrel.

Advantageously, gas and/or liquid may escape from the collected core sample via the fluid flow path(s) provided by the or each side wall.

Known inner barrels typically have a smooth continuous, side wall. Accordingly, the only route for the gas and/or liquid to escape from the collected core sample is via the top and/or bottom of the elongate internal volume.

Typically, the collected core sample may contain gas hydrate, e.g. methane hydrate, crystals. Gas and/or liquid derived from the collected core sample may pass along the or each fluid flow path. Therefore, the gas and/or liquid may have less far to travel through the body of the collected core sample in order to escape from the elongate internal volume. Advantageously, this may help to reduce pressure build-up within the elongate volume. Additionally or alternatively, it may help to reduce the length of time over which gas and/or liquid flows from a given core sample. Consequently, it may be quicker and easier to collect data from a given core sample. Additionally or alternatively, there may be less solid matter, e.g. particles of rock or sediment, entrained in the flow of gas and/or liquid from the collected core sample, since the gas and/or liquid may not have had to pass through as great a volume of the collected core sample.

The or each fluid flow path may comprise one or more at least partially open channels.

In an embodiment, the or each side wall may comprise one or more formations protruding in an inward direction or an outward direction, e.g. radially inwardly or radially outwardly, and bounding at least partially at least a portion of the or each fluid flow path.

In an embodiment, at least a portion of the or each side wall may be fluted, e.g. longitudinally or helically fluted.

The or each side wall may comprise one or more holes or perforations.

The or each fluid flow path may comprise a passageway passing through the or each side wall. For instance, at least a portion of the or each side wall may be porous and/or may contain one or more, e.g. a network of, internal pathways. Additionally or alternatively, at least a portion of the or each side wall may be made from a material through which gas can diffuse, e.g. a polymeric material such as high density polyethylene (HDPE), thereby providing at least a portion of the or each fluid flow path.

In an embodiment, the or each side wall may comprise a plurality of fluid flow paths. For instance, the plurality of fluid flow paths may be regularly spaced from one another. Additionally or alternatively, one or more of the plurality of fluid flow paths may be discrete from the other fluid flow path(s) and/or one or more of the plurality of fluid flow paths may be interconnected with at least one other fluid flow path.

In an embodiment, the or each fluid flow path may allow liquid and/or gas to flow generally sideways from the elongate internal volume and then generally lengthways towards an end of the inner barrel.

In an embodiment, the inner barrel may comprise a first physical separation means arranged to prevent, obstruct or hinder solid particles from entering the or each fluid flow path.

In an embodiment, the first physical separation means may be configured such that solid particles or sediment of a predetermined size cannot enter the or each fluid flow path. For instance, the first physical separation means may be configured such that solid particles or sediment having a smallest dimension of 20 mm or more, e.g. 10 mm or more or 7 mm or more, cannot enter the or each fluid flow path.

The first physical separation means may comprise one or more of a baffle, a relatively narrow inlet to the or each fluid flow path, a filter or a screen. The filter or screen may comprise a mesh or a membrane.

Preventing, obstructing or hindering solid particles, e.g. rock or sediment, from entering the or each fluid flow path may be advantageous, since the or each fluid flow path may be less likely to become blocked, thereby restricting or preventing the flow of liquid and/or gas from the collected core sample. Additionally or alternatively, equipment downstream of the inner barrel may not need to be as complex and/or expensive and/or resilient, since abrasion and/or erosion may be reduced. Advantageously, such downstream equipment may not need to be serviced, maintained or replaced as often.

In an embodiment, the inner barrel may be tubular in form. The inner barrel may have a uniform cross-section along its length.

In an embodiment, the inner barrel may be cylindrical.

In an embodiment, the inner barrel may be made as a complete barrel or tube.

In an embodiment, the inner barrel may comprise a plurality of barrel portions, which may be brought together to form the inner barrel. For instance, the inner barrel may comprise a pair of semi-tubular portions.

The inner barrel or the or each barrel portion may be made as a single piece or may comprise a plurality of pieces, which are joined together, e.g. by welding or an adhesive, or which are configured, e.g. shaped, to fit together without additional mass.

The inner barrel or the or each barrel portion may be formed by extrusion.

The inner barrel or the or each barrel portion may be made from a metal, e.g. a steel, aluminium or an aluminium alloy, or a plastic material, e.g. high density polyethylene.

The inner barrel may have a length of at least 0.5 m, typically at least 1 m. The length of the inner barrel may be up to 5 m, typically up to 4 m or up to 3 m. In an embodiment, the length of the inner barrel may be from 1.5 m to 3 m, e.g. from 1.5 m to 2 m.

The inner barrel may have a maximum width, e.g. an outer diameter, of 0.3 m or more. The maximum width, e.g. outer diameter, of the inner barrel may be up to 1.5 m, e.g. up to 1.2 m. In an embodiment, the maximum width, e.g. outer diameter, of the inner barrel may be at least 0.5 m and/or up to 1 m.

The or each side wall may have a maximum wall thickness of up to 100 mm, e.g. up to 50 mm.

In an embodiment, the inner barrel may comprise a second physical separation means such as a filter or a screen, which extends at least partially across an end of the elongate internal volume. The second physical separation means may be made from a polymeric material, typically HDPE. Advantageously, the second physical separation means may serve to obstruct, hinder or prevent the passage of solid particles entrained in liquid and/or gas, which escapes from the recovered core, through the end of the elongate internal volume. The inner barrel may comprise a second physical separation means across both ends of the elongate internal volume.

In an embodiment, the inner barrel may comprise a third physical separation means located across the outlet from the or each fluid flow path. In embodiments in which the or each fluid flow path terminates at an end of the or each side wall, the third physical separation means conveniently may be part of or joined to the second physical separation means.

In an embodiment, the inner barrel may be provided with a capping system comprising a cap with a fluid flow path therethrough. Optionally or preferably, the capping system may be designed to fail should the pressure inside the inner barrel reach a predetermined value.

A second aspect of the invention provides a core barrel comprising an inner barrel according to the first aspect of the invention and an outer barrel, which provides an impermeable sleeve around the inner barrel.

In an embodiment, the core barrel may be provided with a capping system comprising a cap with a fluid flow path therethrough. Optionally or preferably, the capping system may be designed to fail should the pressure inside the inner barrel reach a predetermined value.

In an embodiment, the capping system may comprise a flowmeter. The flowmeter may be a three-phase flowmeter. The flowmeter may be connected to a data logger and a power supply. For instance, the power supply may comprise an onboard power supply such as a battery.

Alternatively or additionally, the capping system may comprise a contactless connector. Advantageously, the contactless connector may provide, in use, data transmission from the capping system. The contactless connector may be part of an acoustic system or a high frequency system. In an embodiment comprising the contactless connector, there may be no need for an onboard data logger and/or onboard power supply.

A further aspect of the invention provides a method for determining a gas content of a core sample, the method comprising:
taking a core sample from a sediment in a seabed;
storing the core sample in an inner core barrel according to a first aspect of the invention;
lifting the inner core barrel and core sample from the seabed;
measuring an amount of gas released by the lifted core sample; and
determining the gas content of the sediment on the basis of the amount of gas released by the lifted core sample.

In an embodiment, the inner core barrel and core sample may be lifted to a predetermined waterdepth at an ambient pressure at which any gas hydrate crystals in the core sample dissociate into water and gas. The inner core barrel and core sample may be held at the predetermined waterdepth for a period of time, e.g. until very little or no gas is being released by the lifted core sample.

In an embodiment, the inner core barrel and core sample may be lifted to the surface, e.g. on to the deck of a vessel or platform, without holding the inner core barrel and core sample at a predetermined water-depth.

In an embodiment, the amount of gas released may be measured, e.g. continuously, as the inner core barrel and core sample are lifted.

A further aspect of the invention provides a system for determining a gas content of a core sample, the system comprising:
a core sampling device for taking one or more core samples from a sediment in a seabed;
one or more inner core barrels according to the first aspect of the invention for storing the core sample(s);
means for lifting the inner core barrel(s) and core sample(s) from the seabed, e.g. to a predetermined waterdepth at an ambient pressure at which any gas hydrate crystals in the core sample dissociate into water and gas;
a gas sensing device for measuring an amount of gas released by the lifted core sample(s); and
means for determining the gas content of the seabed sediment on the basis of the amount of methane released by the lifted core sample(s)

A further aspect of the invention provides a capping system for a core barrel, the capping system comprising a cap having a fluid flow path therethrough and being configured to fail should the pressure inside the core barrel reach a predetermined value.

In order that the invention may be well understood, exemplary embodiments of the invention will be described with reference to the accompanying drawings, in which:

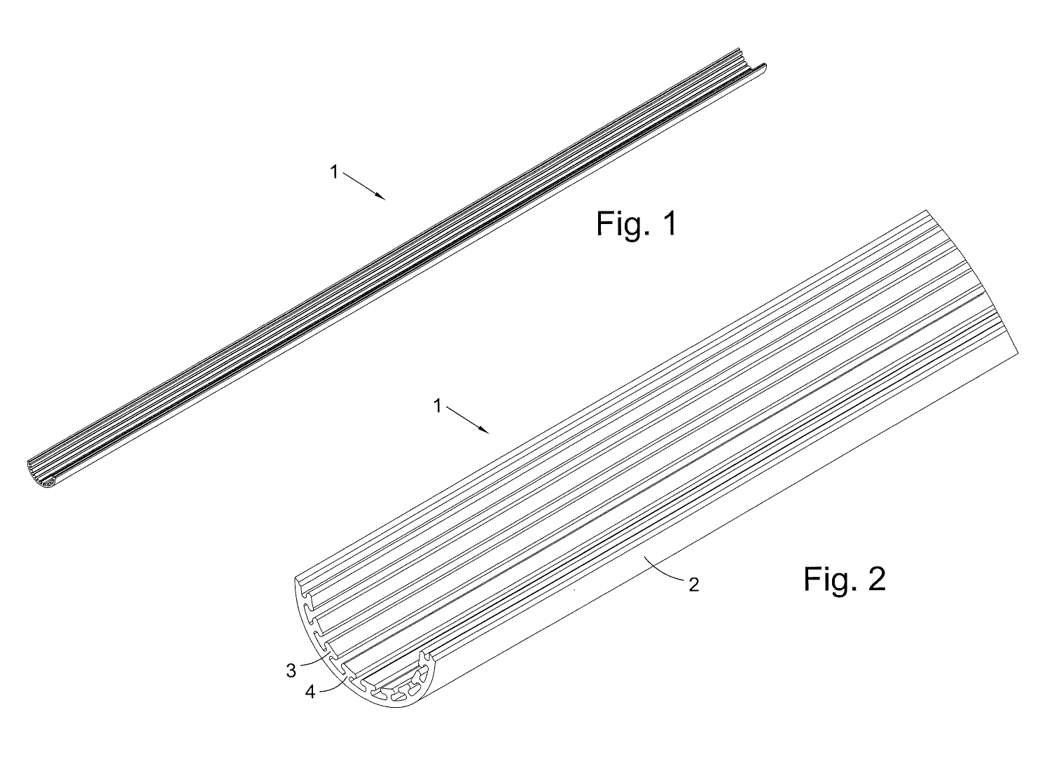

FIG. 1 shows a half of an inner core barrel according to the invention;

FIG. 2 is a larger-scale view of an end portion of the half shown in FIG. 1;

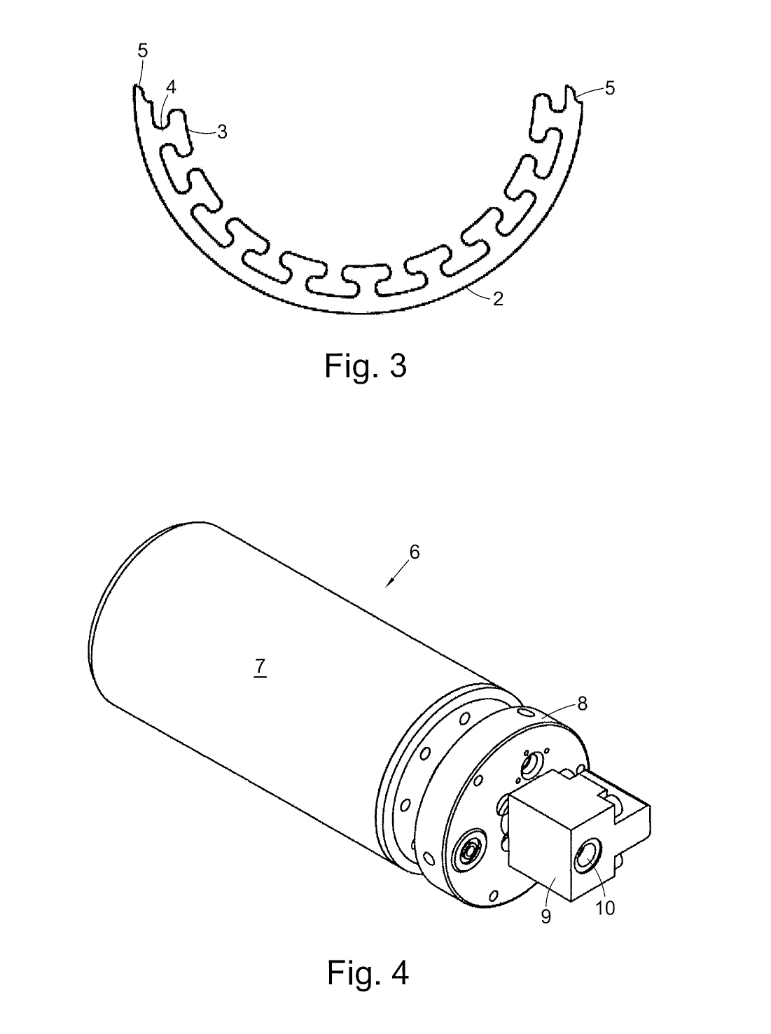

FIG. 3 shows the half of FIG. 1 in cross section;

FIG. 4 is a first isometric view of an exemplary embodiment of acapping system according to the invention;

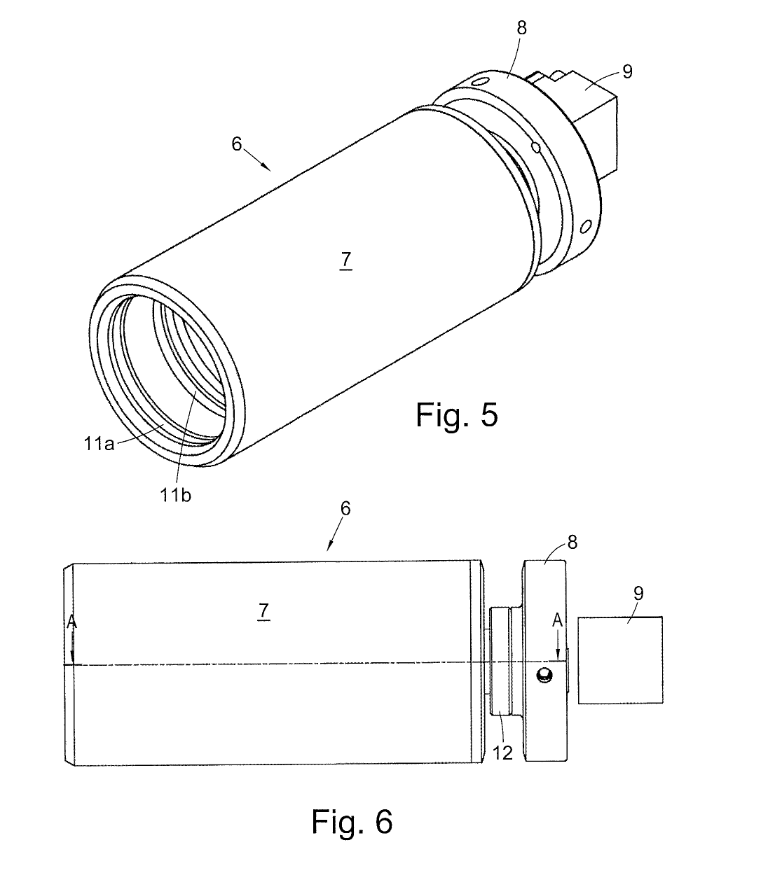

FIG. 5 is a second isometric view of the capping system shown in FIG. 4;

FIG. 6 is an elevation of the capping system shown in FIG. 4;

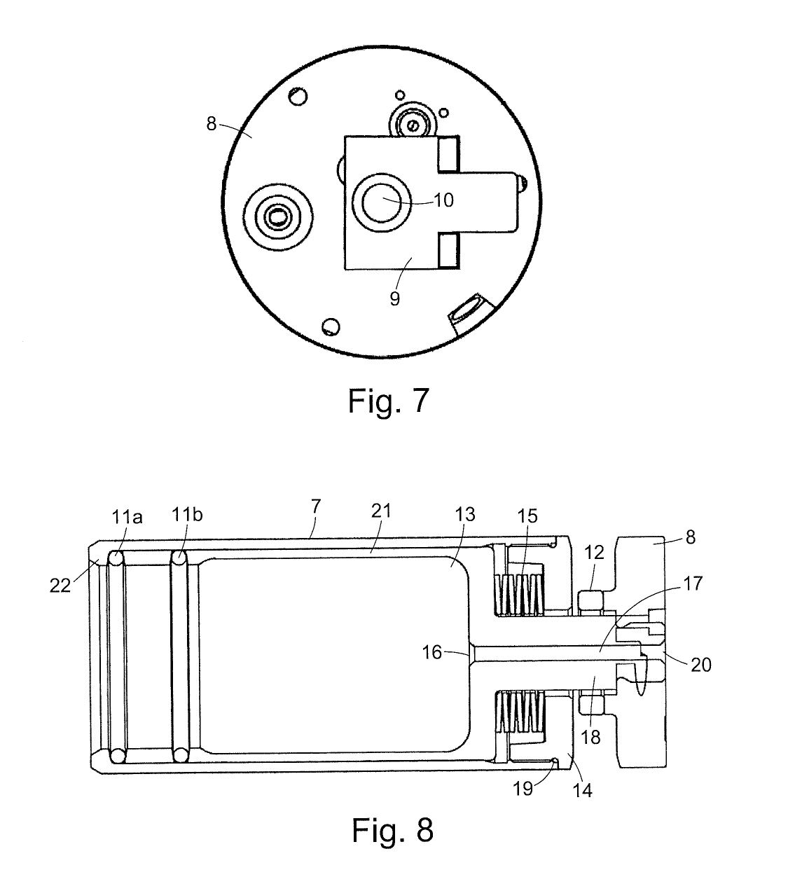

FIG. 7 is a plan view of the capping system shown in FIG. 4;

FIG. 8 is a cross-section along line A-A in FIG. 6;

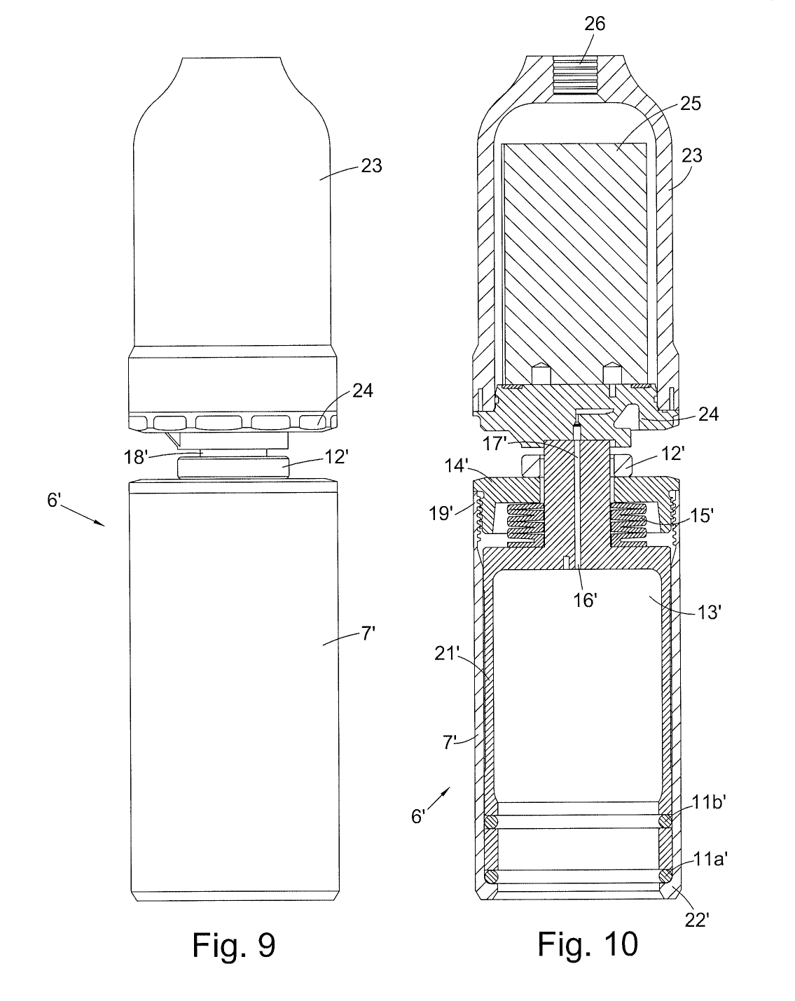

FIG. 9 shows a second exemplary embodiment of a capping system according to the invention; and

FIG. 10 is a longitudinal cross-section of the capping system shown in FIG. 9.

FIG. 1 shows a half 1 of an inner core barrel according to the invention. The half 1 is an aluminium extrusion having a length of 1644 mm. The half 1 is generally semitubular in shape. In use, two halves 1 are brought together to provide a tubular inner core barrel.

FIG. 2 is a larger-scale view of an end portion of the half 1 shown in FIG. 1. The half 1 has a smooth outer surface 2. The inside of the half 1 is fluted longitudinally. Nine regularly spaced generally T-shaped formations, each of which comprises a neck portion 4 and a broader head portion 3, extend along the length of the half 1.

FIG. 3 shows the half 1 in cross-section. The half 1 has an outer radius of 33 mm and an inner radius, measured to the top surfaces of the head portions 3, of 26 mm. The pattern of formations which provide the longitudinal fluting has a repeat distance of 20° of arc. The gap between neighbouring head portions 3 is around 2.4 mm or around 5° of arc. Each head portion 3 has a width of around 15° of arc. Each neck portion 4 has a width of around 5° of arc.

Each end of the half 1 is provided with a curved tongue 5. The curved tongues 5 are shaped and dimensioned such that they mate when two halves 1 are brought together to provide an inner core barrel according to the invention. The curved tongues 5 are simply one example of a suitable shape for each end of the half. Many other suitable shapes, e.g. linear, curved or curvilinear shapes, will be apparent to the person skilled in the art. What is important is that the ends of the barrel portions, e.g. halves, mate when the barrel portions, e.g. two halves, are brought together to provide an inner core barrel according to the invention.

In use, two halves 1 are brought together to form an inner core barrel according to the invention. The longitudinal fluting provides a plurality of fluid flow paths for gas released from a core sample held within the inner core barrel. Gas can flow radially outwards from the core sample at substantially any point along the length of the core sample by passing through the gaps between the head portions 3. The gas may then flow along the length of the inner core barrel in channels between the formations. In addition, the relatively small width of the gap between the head portions 3, as compared with the gap between neighbouring neck portions 4 underneath the head portions 3, serves to hinder or prevent any large solid particles, e.g. rock or sediment, from entering the fluid flow channels with the gas.

In use, an inner core barrel according to the invention may be housed within an outer core barrel. The combination of an outer core barrel with an inner core barrel therein may be termed a core barrel.

FIGS. 4, 5 and 6 show an exemplary embodiment of a capping system 6 according to the invention for sealing an end of an inner core barrel or a core barrel according to the invention. The capping system 6 comprises an open-ended housing 7 shaped and dimensioned to receive an end of a core barrel.

As can be seen in FIG. 5, the inside of the housing 7 is provided with a pair of sealing rings 11a, 11b located relatively close to the open end of the housing 7. In use, the sealing rings 11a, 11b form an air-tight seal with an outer surface of the core barrel received within the housing 7.

The capping system also comprises a top cap 8 and an instrument block 9 housing a flowmeter. As will be described later, fluid flows, in use, from the inner volume of the housing 7 though a neck portion 12 of the top cap 8 and through the instrument block 9.

FIG. 7 is a plan view showing the instrument block 9 on top of the top cap 8. The instrument block has an outlet 10, which can be connected to further tubing (not shown) leading to a drilling platform or vessel at the surface.

The instrument block 9 contains a flowmeter for measuring the flow of fluid from a core sample. The flowmeter is connected to a data logger and a battery to supply power to the flowmeter and/or the data logger. The data logger and/or the battery may be housed within the instrument block 9. The flowmeter may be a coriolis flowmeter. The flowmeter may be a three-phase flowmeter for measuring the flow of and distinguishing between liquid, gas and mixtures of liquid and gas. The flowmeter may be supplied by Bronkhurst.

The data logger may be associated with a pressure sensor arranged such that power is supplied from the battery only when the ambient pressure is within a predetermined range. This may help to prolong batter life. Accordingly, the data logger can be armed at the seabed or the surface, but will only begin to operate at the predetermined ambient pressure. The predetermined ambient pressure is selected to correspond to a pressure at which gas hydrates will dissociate to gas and water.

Alternatively or additionally, power may be supplied to the data logger from the surface. A data link, e.g. a cable or a wireless or contactless connection such as an acoustic or high frequency connection, may also be provided from the data logger to a surface facility, thereby allowing real-time analysis of flow data.

FIG. 8 is a cross-section along line A-A in FIG. 6. The housing 7 contains an inner sleeve 21. The inner sleeve 21 fits inside the housing 7 from the top and is prevented from sliding out of the bottom of the housing 7 by a lip 22. The opening of the housing 7 is slightly narrower than an internal cavity 13. In use, the sealing rings 11a, 11b provide an air-tight seal with an outer surface of a core barrel (not shown). An end of the core barrel is then located within the internal cavity 13.

The internal cavity 13 has an outlet 16 located centrally on its end surface. The outlet 16 leads to a passageway 17, which passes through a long neck portion 18 of the housing 7. The neck portion 12 of top cap 8 surrounds an end portion of the long neck portion 18 of the housing 7. The passageway 17 extends into and through the top cap 8 to an outlet 20, to which the instrument block 9 is connected, in use.

The capping system 6 provides fluid flow from a core sample in a core barrel received within the housing 7 to a flowmeter. The capping system is also designed to fail should the internal pressure within the housing 7 reach a predetermined value. A spring 15 or other resilient biasing means is provided between an outer surface of the top of the internal cavity 13 and an underside of a closure member 14 disposed around the neck portion 18 and below the neck portion 12. The closure member 14 is held in place by a lip 19 on the housing 7. If there is a pressure build-up within the internal cavity 13, then the sleeve 21 is forced upwards against the spring 15, which in turn forces the closure member 14 out of the housing 7. Gas can then escape from the internal cavity 13.

FIGS. 9 and 10 show a second exemplary embodiment of a capping system 6′ according to the invention for sealing an end of an inner core barrel or a core barrel according to the invention. The capping system 6′ comprises an open-ended housing 7′ shaped and dimensioned to receive an end of a core barrel.

At its closed end, the housing 7′ is connected to an instrument housing 23 containing a flowmeter (not shown). Fluid flows, in use, from the inner volume of the housing 7′ through a neck portion 12′, a long neck portion 18′ and a base portion 24 to an internal volume 25 of the instrument housing 23.

As can be seen in FIG. 10, the inside of the housing 7′ is provided with a pair of sealing rings 11a′, 11b′ located relatively close to the open end of the housing 7′. In use, the sealing rings 11a′, 11b′ form an air-tight seal with an outer surface of the core barrel received within the housing 7′.

The housing 7′ contains an inner sleeve 21′. The inner sleeve 21′ fits inside the housing 7′ from the top and is prevented from sliding out of the bottom of the housing 7′ by a lip 22′. The opening of the housing 7′ is slightly narrower than an internal cavity 13′. In use, the sealing rings 11a′, 11b′ provide an air-tight seal with an outer surface of a core barrel (not shown). An end of the core barrel is then located within the internal cavity 13′.

The internal cavity 13′ has an outlet 16′ located centrally on its end surface. The outlet 16′ leads to a passageway 17′, which passes through a long neck portion 18′ of the housing 7′. The neck portion 12′ surrounds an end portion of the long neck portion 18′ of the housing 7′. The passageway 17′ extends into the base portion 24 of the instrument housing 23.

The capping system 6′ provides fluid flow from a core sample in a core barrel received within the housing 7′ to a flowmeter located in the internal volume 25 of the instrument housing 23. The capping system 6′ is also designed to fail should the internal pressure within the housing 7′ reach a predetermined value. A spring 15′ or other resilient biasing means is provided between an outer surface of the top of the internal cavity 13′ and an underside of a closure member 14′ disposed around the long neck portion 18′ and below the neck portion 12′. The closure member 14′ is held in place by a lip 19′ on the housing 7′. If there is a pressure build-up within the internal cavity 13′, then the sleeve 21′ is forced upwards against the spring 15′, which in turn forces the closure member 14′ out of the housing 7′. Gas can then escape from the internal cavity 13′.

The instrument housing 23 has an outlet 26, which can be connected to further tubing (not shown) leading to a drilling platform or vessel at the surface. As shown in FIG. 10, the outlet 26 is threaded, so that the instrument housing 23 can be screwed onto an end of a threaded tube or pipe. The instrument housing 23 may be configured to be connectable to further tubing by any suitable means.

The internal volume 25 of the instrument housing 23 contains a flowmeter for measuring the flow of fluid from a core sample. The flowmeter is connected to a data logger and a battery to supply power to the flowmeter and/or the data logger. The data logger and/or the battery may be housed within the instrument housing 23. The flowmeter may be a coriolis flowmeter. The flowmeter may be a three-phase flowmeter for measuring the flow of and distinguishing between liquid, gas and mixtures of liquid and gas. The flowmeter may be supplied by Bronkhurst.

The data logger may be associated with a pressure sensor arranged such that power is supplied from the battery only when the ambient pressure is within a predetermined range. This may help to prolong batter life. Accordingly, the data logger can be armed at the seabed or the surface, but will only begin to operate at the predetermined ambient pressure. The predetermined ambient pressure is selected to correspond to a pressure at which gas hydrates will dissociate to gas and water.

Alternatively or additionally, power may be supplied to the data logger from the surface. A data link, e.g. a cable or a wireless or contactless connection such as an acoustic or high frequency connection, may also be provided from the data logger to a surface facility, thereby allowing real-time analysis of flow data.

An example of a method according to the invention will now be described.

A core sampling device is operated on the seabed to collect a plurality of core samples. The core sampling device is operable to transfer each core sample to a core barrel comprising an inner core barrel according to the invention and an outer barrel which serves as impermeable sleeve. A capping system according to the invention is then placed on the top end of each core barrel. The bottom end of each core barrel is also sealed, e.g. with a cap.

The combination of a core barrel and a capping system may be termed a core barrel assembly.

When all of the core barrel assemblies carried by the core sampling device contain a core sample, the core sampling device is lifted from the seabed to a predetermined depth. Typically, the predetermined depth is above the Gas Hydrate Stability Zone (GHZ).

The core sampling device is held at the predetermined depth while any gas hydrate crystals in the core sample dissociate into gas and water.

Each capping system is equipped with a flowmeter and a data logger. Accordingly, the amount of gas escaping from each core sample is measured and recorded individually. Providing a flowmeter and a data logger for each core sample held in the core sampling device may enable more accurate data to be gathered.

Having passed through the flowmeter, the gas flows up a tube or conduit to the surface.

When the amount of gas flowing through the flowmeters suggests that all or substantially all of the gas has escaped from the core samples, the core sampling device is recovered to the surface, e.g. to a drilling platform or a vessel. The collected core samples may then be taken away for further analysis.

The capping system may be attached to the core barrel assembly at the surface, then taken off at the seabed, in order to place a collected core sample in the core barrel assembly, before being put back on ahead of lifting the core sampling device from the seabed.

The method may be repeated at a plurality of different locations in order to survey an area of seabed. Such a survey can be carried out relatively quickly and cheaply, particularly when compared with pressurised core barrel techniques. Typically, a survey may take 15 days per core sample using a technique involving bringing a pressurised core barrel to the surface. In contrast, a survey employing the apparatus and methods of the present invention may take significantly less time, typically around 24 hours per hole or site. Thus, the invention may permit an area of seabed to be mapped rapidly and accurately.

Use of the inner core barrel and/or the capping assembly according to the invention may improve the speed and accuracy of the method described in WO 2011/082870. For instance, it may enable the distribution of hydrate across a give region to be determined and/or mapped more quickly and accurately.

It may be desired to obtain oriented core samples. For instance, a corer as described in GB2465829 may be used to collect multiple oriented cores before retrieval of the corer to the surface.

In some embodiments, the core samples may be brought to the surface before all of the gas has escaped. Further measurement and analysis may be carried out at the surface while the gas hydrate fully dissociates.

In some embodiments, the core samples may not be held at a predetermined depth. They may be brought directly to the surface for measurement and analysis. If the core samples are brought to the surface directly, then there may be no need to provide a flowmeter and data logger within the capping system.

Rather than providing a flowmeter and data logger for each core sample of a plurality of core samples collected by a core sampling device, gas flow from all of the core samples may be measured together by a single flowmeter and data logger located downstream of a junction uniting tubing from all of the core barrel assemblies.

The capping system may be provided with a stab point, in case the core barrel assembly needs to be depressurised at the surface.

In another method according to the invention, the core sample(s) may be brought up the water column, whilst logging the flow of gas and fluid against water depth and/or ambient pressure.

1. An inner barrel for a core barrel or a core barrel assembly, the inner barrel having one or more side walls bounding at least partially an elongate internal volume for receiving, in use, a collected core sample, wherein the or each side wall is adapted to provide at least one fluid flow path from the elongate internal volume to outside the inner barrel.

2. An inner barrel according to claim 1, wherein the or each fluid flow path comprises:
one or more at least partially open channels. or
a passageway passing through the or each side wall.

3. An inner barrel according to claim 1, wherein the or each side wall comprises one or more formations protruding in an inward direction or an outward direction, e.g. radially inwardly or outwardly, and bounding at least partially at least a portion of the or each fluid flow path.

4. An inner barrel according to claim 1, wherein:
(i) at least a portion of the or each side wall is fluted; and/or
(ii) the or each side wall comprises a plurality of fluid flow paths.

5. (canceled)

6. (canceled)

7. (canceled)

8. An inner barrel according to claim 1, wherein the or each fluid flow path allows liquid and/or gas to flow generally sideways from the elongate internal volume and then generally lengthways towards an end of the inner barrel.

9. An inner barrel according to claim 1, further comprising a first physical separation means arranged to prevent, obstruct or hinder solid particles from entering the or each fluid flow path.

10. An inner barrel according to claim 9, wherein the first physical separation means is configured such that solid particles or sediment of a predetermined size cannot enter the or each fluid flow path.

11. An inner barrel according to claim 9, wherein the first physical separation means comprises one or more of a baffle, a relatively narrow inlet to the or each fluid flow path, a filter or a screen.

12. (canceled)

13. An inner barrel according to claim 1, wherein the inner barrel comprises a plurality of barrel portions, which may be brought together to form the inner barrel.

14. An inner barrel according to claim 9, further comprising a second physical separation means such as a filter or a screen, which extends at least partially across an end of the elongate internal volume.

15. (canceled)

16. A core barrel assembly comprising an inner core barrel according to claim 1, and a capping system comprising a cap with a fluid flow path therethrough.

17. A core barrel assembly according to claim 16, wherein the capping system is designed to fail should the pressure inside the inner barrel reach a predetermined value.

18. A core barrel assembly according to claim 17, wherein the capping system comprises a flowmeter.

19. A core barrel assembly according to claim 18, wherein the flowmeter is a three-phase flowmeter, and/or wherein the flowmeter is connected to a data logger and a power supply.

20. (canceled)

21. A method for determining a gas content of a core sample, the method comprising:
taking a core sample from a sediment in a seabed;
storing the core sample in an inner core barrel according to claim 1;
lifting the inner core barrel and core sample from the seabed; measuring an amount of gas released by the lifted core sample; and
determining the gas content of the sediment on the basis of the amount of gas released by the lifted core sample.

22. A method according to claim 21, wherein the inner core barrel and the core sample are lifted to a predetermined waterdepth at an ambient pressure at which any gas hydrate crystals in the core sample dissociate into water and gas.

23. A method according to claim 21, wherein the core sample is an oriented core sample.

24. A system for determining a gas content of a core sample, the system comprising:
a core sampling device for taking one or more core samples from a sediment in a seabed;
one or more inner core barrels according to any claim 1 for storing the core sample(s);
means for lifting the inner core barrel(s) and core sample(s) from the seabed;
a gas sensing device for measuring an amount of gas released by the lifted core sample(s); and
means for determining the gas content of the seabed sediment on the basis of the amount of gas, e.g. methane, released by the lifted core sample(s).

25. A system according to claim 24, wherein:
(i) the gas sensing device comprises a flowmeter; and/or.
(ii) the core sampling device is operable to take oriented core samples.

26. A system according to claim 25 further comprising a data logger connected to the flowmeter.

27. (canceled)
